(19) United States
(12) Patent Application Publication
(10) Pub. No.: US 2004/0223928 A1
(43) Pub. Date: Nov. 11, 2004

(54) Cosmetic compositions for skin

(75) Inventors: Teresa Barbara Crook, Camberley (GB); Paul Jonathan Matts, Addlestone (GB); Alison Fiona Stephens, Cookham (GB)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/840,832

(22) Filed: May 7, 2004

Related U.S. Application Data
(60) Provisional application No. 60/468,952, filed on May 8, 2003.

(51) Int. Cl. A61K 7/021

(52) U.S. Cl. 424/63

(57) ABSTRACT

Topical compositions comprising gold particulates and silver particulates that impart unique colour and reflectance changes to the skin when applied are provided.

### CROSS REFERENCE TO RELATED APPLICATION

This application claims the benefit of U.S. Provisional Application No. 60/468,952, filed May 8, 2003

### FIELD OF THE INVENTION

The present invention relates to topical compositions for application to the skin. More specifically, topical compositions are provided that comprise gold and silver particulate material. The topical compositions of the present invention provide increased skin tone and lustre with excellent skin application and moisturization.

### BACKGROUND OF THE INVENTION

Topical compositions are well known and widely used. These compositions have long been employed to cleanse and moisturize skin, deliver actives, hide imperfections and to reduce the oiliness/shine associated with sebum. Topical compositions have also been used to alter the colour and appearance of skin.

These compositions generally incorporate organic or inorganic particulate material to reduce the shine or redness of skin, and to also cover over skin imperfections such as wrinkles. For example, emulsions may contain TiO₂ as an opacifying agent to provide a white appearance to the emulsion. Several publications have also disclosed the use of TiO₂ in topical compositions. See, e.g. U.S. Pat. No. 5,223,559 and JP 08188723. In addition, R. Emmert has stated the desire to use optical means to formulate products that give the consumer an immediate, visual improvement (Dr. Ralf Emmert, Quantification of the Soft-Focus Effect, Cosmetics & Toiletries, Vol. 111, July 1996, pp. 57-61). Emmert discloses that one can mechanically fill in skin lines with a reflective substance such as TiO₂. However, Emmert teaches that such reflective materials result in an undesirable mask-like appearance, and that one should therefore use a material that diffuses light yet is sufficiently transparent to avoid the mask-like appearance.

Previous topical compositions containing reflective materials tend to result in an unnatural appearance when applied to the skin. WO 00/51551 teaches the use of low levels of green interference pigment in topical compositions to offset areas of redness in the skin, whilst U.S. Pat. No. 5,972,359 teaches that particulate materials having a refractive index of at least 2 and a primary particle size of from 100 nm to 300 nm are useful in topical compositions for regulating visible and/or tactile discontinuities in skin. The green interference pigment disclosed in WO 00/51551 can result in a composition that is not suitable on all skin types. The particles disclosed in WO 00/51551, whilst being very effective in reducing the appearance of skin imperfections, can result in the skin appearing matte, white and ashy due to the shape and high refractive index of the particulate materials incorporated therein.

Therefore, it is desirable to provide a topical composition comprising a select level and blend of gold and silver particulates to provide a unique level of light reflectance and colour shift to increase the shine across all skin types. Furthermore, it is desirable to provide a topical composition comprising both particulates and chronic texture-regulating agents that act synergistically to maximise sheen and lustre on the skin. It is desirable to include chronic texture regulating agents to smooth any discontinuities such as wrinkles. It is further desirable to provide topical compositions that effectively reduce the appearance of wrinkles whilst providing skin moisturization.

### SUMMARY OF THE INVENTION

Topical compositions comprising gold-coloured particulates and silver-coloured particulates are provided, wherein the compositions, when measured at 15° from specular 15 minutes after topical application of 2□l/cm² in vivo, have a reflectance of less than about 200 and a delta E value of less than about 4. The topical compositions of the present invention provide a well-balanced shine and colour to the skin when applied that matches and highlights the skin's natural tones. Furthermore, topical compositions comprising gold and silver particulates and chronic skin texture-regulating agents are provided.

### BRIEF DESCRIPTION OF THE DRAWINGS

FIG. 1 is a graph comparing the increase in reflectance for a number of comparative compositions and embodiments of the present invention.

FIG. 2 is a graph comparing the change in colour (delta E value) for a number of comparative compositions and embodiments of the present invention.

### DETAILED DESCRIPTION OF THE INVENTION

All weights, measurements and concentrations herein are measured at 25° C. on the composition in its entirety, unless otherwise specified.

Unless otherwise indicated, all percentages of compositions referred to herein are weight percentages and all ratios are weight ratios.

Unless otherwise indicated, all molecular weights are weight average molecular weights.

Unless otherwise indicated, the content of all literature sources referred to within this text are incorporated herein in full by reference.

Except where specific examples of actual measured values are presented, numerical values referred to herein should be considered to be qualified by the word “about”.

Herein, “gold-coloured particulates” includes particulate materials that reflect gold-coloured light.

Herein, “silver-coloured particulates” includes particulate materials that reflect silver-coloured light.

Active and other ingredients useful herein may be categorised or described herein by their cosmetic and/or therapeutic benefit or their postulated mode of action. However, it is to be understood that the active and other ingredients useful herein can in some instances provide more than one cosmetic and/or therapeutic benefit or operate via more than one mode of action. Therefore classifications herein are made for the sake of convenience and are not intended to limit an ingredient to the particularly stated application or applications listed.

The topical compositions of the present invention comprise gold-coloured particulates and silver-coloured particulates. These particulates are present in levels and blends that provide a light reflectance and colour shift that is highly acceptable to consumers when applied to the skin. The compositions of the invention are useful for topical application and for providing essentially immediate (i.e. acute) improvement in skin appearance following topical application. Without being limited by theory, it is believed that this acute improvement results at least in part from an immediate increase in the reflectance of light from the skin by the particulate materials without unnatural whitening of the skin. The combination of light reflectance and colour shift (delta E value) create a composition that has very good acute benefits on application, namely producing good skin shine due to reflected light without the skin appearing unnatural or oily.

Herein, reflectance and delta E are measured on the whole composition in vivo on human skin using the X-Rite MA68 II, 5-angle spectrophotometer, an industry standard device for analysing reflected light and colour. The spectrophotometer incident light source is at 45° to the surface, and it analyses reflected light at 15°, 25°, 45°, 75° and 100° away from specular. A base size of at least 25 individuals is required to provide accurate data. Prior to use the instrument should be calibrated using the white and black standards supplied with the machine.

The skin is illuminated with incident light at 45° from normal. The light source is a D₆₅ illuminant at a 10° standard observer. The skin is read without product to provide a base-line measurement. The product is then applied to bare skin without markings such as tattoos on the forearm at a dosage of 2 μl/cm² with the area required to be greater than the instrument capture port size which is a circle of diameter 12 mm. An area of 2 cm×2 cm square (4 cm²) is sufficient. The product should be applied in an even film and rubbed until absorbed (30 seconds). The product is then allowed to dry on the skin for 15 minutes. Application on skin in vivo is required as some materials in the product may enhance the reflectance and delta E values initially, but are subsequently absorbed into the skin, producing a more matte finish. Baseline measurements are subtracted from post-application readings for each subject to provide information on changes due to the product. Data can be analysed using QA Master 2000 software supplied with the X-Rite.

The equipment provides measurements of the reflectance (as a percentage of the inputted light) at each of the 5 angles over the full visible range of 400-700 mn (measured at 10 nm band-widths). To get the reflectance as a function of angle, the reflectance at each band-width are summed, and then the base-line measurement for each angle is subtracted to give the change in reflectance. As will be appreciated, for highly matte materials, this number may be negative, as these materials will actually reduce the amount of reflected light. Herein, “reflectance” means the increase in reflected light over the baseline measurement of bare skin at 150 from specular 15 minutes following topical application of 2□l/cm² in vivo.

The equipment will also provide measurements of the 3D colour co-ordinate measurements (L, a and b) as a function of angle. The delta E values are then calculated using the following equation:

$$Delta E = \sqrt{[ {( L - {L}^{*} )}^{2} + {( a - {a}^{*} )}^{2} + {( b - {b}^{*} )}^{2} ]}$$

Wherein, L, a and b are the 3D colour co-ordinate measurements for the composition on the skin, and L*, a* and b* are the 3D colour co-ordinate measurements for the bare skin. Herein, the delta E values are taken as the change in 3D colour-co-ordinate measurements at 15° from specular 15 minutes following topical application of 2□l/cm² in vivo.

The topical compositions herein provide an essentially immediate (acute) visual improvement in skin appearance that is generated by the topical application of the gold-coloured and silver-coloured particulate materials. Without being bound by theory, it is believed that this acute skin appearance improvement results, at least in part, from the reflection of gold and silver light from the skin.

The topical compositions herein have a reflectance of less than about 200, preferably from about 10 to about 200, more preferably from about 20 to about 100, more preferably still from about 25 to about 60. It has been found that these levels of reflectance provide a level of reflected light that is neither too shiny nor too matte. Compositions having reflectance values above 200 are perceived as making the skin appear oily or unnatural. Compositions having a reflectance value that is too low (i.e. below 10) do not provide the acute skin radiance benefits that are beneficial to the topical cosmetic compositions of the present invention.

Furthermore, the topical compositions herein have delta E values of less than about 4, preferably from about 1 to about 3.5, more preferably from about 2.0 to about 3.0. It has surprisingly been found that products having these levels of colour shift on human skin provide a limited change in skin tone and colour that creates a healthy appearance on all skin types. Furthermore, the colour change provides excellent acute benefits with regards to the appearance of the skin, without the skin appearing unnaturally coloured or whitened. This whitening can result in darker skin types appearing “ashen” or grey. Compositions having this unique combination of reflectance and delta E provide excellent skin radiance benefits across all skin types.

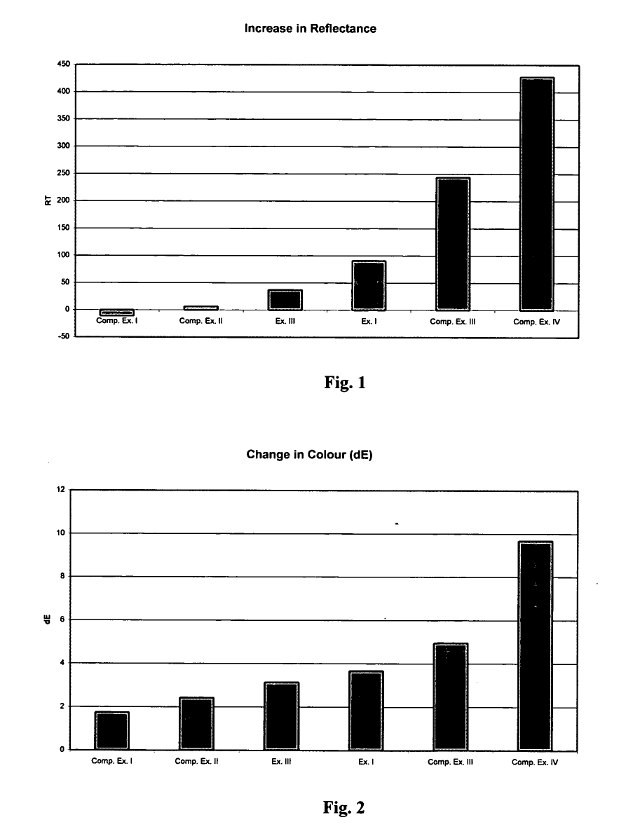

Referring to the figures now, it can clearly be seen in FIGS. 1 and 2 that the compositions of the present invention clearly increase the reflectance and the delta E value of the skin in combination to levels that the comparative compositions are unable to do. The comparative compositions are commercially available compositions, and are listed below, along with an estimated list of ingredients:

Comparative Ex. I: Vaseline™ Intensive Care Dry Skin Hand and Body Lotion (Batch No. 07142J00)

Comparative Ex. II: Composition of Ex. 1 without the gold and silver particulates (Additional water added);

Comparative Ex. III: Nivea™ Silky Shimmer Lotion (Batch No. 13111051)

Comparative Ex. IV: Ellen Betrix™ Perfect Eye Shadow, Pearl Biege (Batch No. 8A1)

Of particular interest is the comparison between the examples of the present invention and comparative examples I and II, neither of which contain any particulates. It can clearly be seen that comparative examples I and II do not increase the reflectance of the skin significantly, indeed comparative example I actually mattefies the skin, resulting in a decreased reflectance. At the other end of the spectrum are comparative examples III and IV, which impart too high a reflectance and colour change on the skin. These compositions generally impart an unnatural shininess on the skin that consumers appear to dislike.

Furthermore, preferred embodiments of the invention are also useful in providing long-term (chronic) improvement in skin appearance by smoothing the texture of the skin using texture-regulating agents. Without wishing to be bound by theory, it is believed that the combination of acute and chronic skin benefits act synergistically to provide an increase in skin smoothness, tone and colour.

The topical compositions of the present invention comprise gold-coloured particulates and silver-coloured particulates. The gold-coloured particulates comprise particulate materials that reflect a gold colour such as gold flakes, composite materials, coated glass, coated micas, and mixtures thereof. The silver-coloured particulates comprise particulate materials that reflect a silver colour such as silver flakes, composite materials, coated glass, coated micas, and mixtures thereof. Preferably, the gold-coloured particulates and silver-coloured particulates useful in the present invention comprise a substrate with at least one interference layer there upon. The interference layer preferably comprises TiO₂, SnO or mixtures thereof. Preferably, the gold-coloured particulates and silver-coloured particulates comprise a platelet-shaped substrate. The substrate comprises mica, a flake of glass, BaSO₄, SiO₂, a synthetic ceramic or mixtures thereof, preferably mica.

Preferably the gold-coloured particulates and silver-coloured particulates are dispersed in the composition. Without wishing to be bound by theory, it is believed that compositions comprising these particulate materials provide excellent reflection of gold/silver light from the skin. Use of particulates reflecting different colours has been found to be less effective in providing colouring on the skin that is acceptable on all skin ethnicities and provides a healthy appearance on the skin.

The topical compositions of the present invention preferably comprise from about 0.1% to about 5% gold-coloured particulates, and from about 0.1% to about 5% silver-coloured particulates. More preferably the topical compositions comprise from about 0.2% to about 2.5% gold-coloured particulates and about 0.2% to about 2.5% silver-coloured particulates, more preferably still from about 0.2% to about 0.5% gold-coloured particulates and from about 0.2% to about 0.5% silver-coloured particulates. Preferably, the ratio of gold-coloured particulates to silver-coloured particulates is from about 5:1 to about 1:5, more preferably from about 2:1 to about 1:3, more preferably still from about 1:1 to about 1:2. It has been found that incorporating these levels and ratios of gold-coloured particulates and silver-coloured particulates in topical compositions provides compositions having the correct reflectance and delta E values that provide immediate coverage of skin imperfections whilst imparting a natural shine and increase in skin tone that is pleasing to the consumer.

The colour of the reflected light varies depending on the thickness of the interference layer over the substrate. The gold-coloured particulates used in the present invention preferably comprise a particulate material having an interference layer thickness of from about 60 nm to about 80 nm. The silver-coloured particulates used in the present invention preferably comprise a particulate material having an interference layer thickness of from about 40 nm to about 59 nm.

Preferably the gold-coloured particulates and silver-coloured particulates herein have a diameter of from about 5 μm to about 75 μm, more preferably from about 15 μm to about 70 μm. Herein, “diameter” means the largest distance across the major axis of the particulate material. Diameter can be determined by any suitable method known in the art, such as ASTM Designation E20-85 “Standard Practice for Particle Size Analysis of Particulate Substances in the range of 0.2 to 75 Micrometers by Optical Microscopy”, ASTM Volume 14.02, 1993. Non-limiting examples of gold-coloured particulates suitable for use herein include Prestige Gold and Bright Gold supplied by Eckart, Flamenco Super Gold, Summit Gold supplied by Engelhard Corporation and Timiron Super Gold and Silk Gold supplied by Merck. Non-limiting examples of silver-coloured particulates suitable for use herein include Prestige Bright Silver and Bright Silver Star also supplied by Eckart, Flamenco Super Silver supplied by Engelhard and Timiron Super Silver by Merck.

The composition of the present invention may further comprise a skin texture-regulating agent. The skin texture-regulating agent comprises from about 0.1% to about 12%, preferably from about 0.25% to about 7%, more preferably from about 2% to about 6% by weight of the composition. Skin texture-regulating agents useful herein comprise vitamin B₃ compounds, panthenol and its derivatives, retinoids and their derivatives, humectants, amino acids and their derivatives, vitamin C and its derivatives and mixtures thereof. Skin texture-regulating agents are useful for providing visual improvements in skin appearance or condition following multiple topical applications of the composition to the skin. This “chronic” benefit is particularly desirable as it evens the texture of skin and so increases skin sheen by providing a desquamatory, keratolytic and rejuvenating effect and/or moisturization. The compositions provide long-term visual benefits in conjunction with immediate improvement of skin appearance without imparting unacceptable skin appearance such as skin whitening or excessive shininess.

One class of skin texture-regulating agent according to the present invention includes vitamin B₃ compounds. As used herein, “vitamin B₃ compound” includes compounds having the formula:

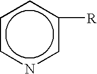

wherein R is —CONH₂ (i.e., niacinamide), —COOH (i.e., nicotinic acid) or —CH₂OH (i.e., nicotinyl alcohol); derivatives thereof; and salts of any of the foregoing. Exemplary derivatives of the foregoing vitamin B₃ compounds include nicotinic acid esters, including non-vasodilating esters of nicotinic acid, nicotinyl amino acids, nicotinyl alcohol esters of carboxylic acids, nicotinic acid N-oxide and niacinamide N-oxide.

Suitable esters of nicotinic acid include nicotinic acid esters of C₁-C₂₂, preferably C₁-C₁₆, more preferably C₁-C₆ alcohols. The alcohols are suitably straight-chain or branched chain, cyclic or acyclic, saturated or unsaturated (including aromatic), and substituted or unsubstituted. The esters are preferably non-vasodilating. As used herein, “non-vasodilating” means that the ester does not commonly yield a visible flushing response after application to the skin in the subject compositions (the majority of the general population would not experience a visible flushing response, although such compounds may cause vasodilation not visible to the naked eye). Non-vasodilating esters of nicotinic acid include tocopherol nicotinate and inositol hexanicotinate; tocopherol nicotinate is preferred. The vitamin B₃ compound is preferably used in an amount of from about 0.1% to about 10%, more preferably from about 2% to about 5%.

A further class of skin texture-regulating agent according to the present invention comprises panthenol or its derivatives. The panthenol and its derivatives include D-panthenol ([R]-2,4-dihydroxy-N-[3-hydroxypropyl)]-3,3-dimethylbutamide), DL-panthenol, calcium pantothenate, royal jelly, panthetine, pantotheine, panthenyl ethyl ether, pangamic acid, pyridoxin, pantoyl lactose and Vitamin B complex. The compositions of this invention may contain a safe and effective amount of the panthenol, such that the resultant composition is safe and effective for regulating skin texture. The panthenol derivative is preferably used in an amount of from about 0.1% to about 5%, more preferably from about 0.2% to about 3%.

A further class of skin texture-regulating agents useful herein comprises retinoids. As used herein, “retinoid” includes all natural and/or synthetic analogs of Vitamin A or retinal-like compounds which possess the biological activity of Vitamin A in the skin as well as the geometric isomers and stereoisomers of these compounds. The retinoid is preferably retinal, including retinol esters (e.g., C₂-C₂₂ alkyl esters of retinol, including retinyl palmitate, retinyl acetate, retinyl proprionate), retinal, and/or retinoic acid (including all-trans retinoic acid and/or 13-cis-retinoic acid), more preferably retinoids other than retinoic acid. These compounds are well known in the art and are commercially available from a number of sources, e.g., Sigma Chemical Company (St. Louis, Mo.), and Boerhinger Mannheim (Indianapolis, Ind.). Other retinoids which are useful herein are described in U.S. Pat. Nos. 4,677,120, issued Jun. 30, 1987 to Parish et al.; U.S. Pat. No. 4,885,311, issued Dec. 5, 1989 to Parish et al.; U.S. Pat. No. 5,049,584, issued Sep. 17, 1991 to Purcell et al.; U.S. Pat. No. 5,124,356, issued Jun. 23, 1992 to Purcell et al.; and U.S. Re. Pat. No. 34,075, issued Sep. 22, 1992 to Purcell et al. Other suitable retinoids are tocopheryl-retinoate [tocopherol ester of retinoic acid (trans- or cis-), adapalene {6-[3-(1-adamantyl)-4-methoxyphenyl]-2-naphthoic acid}, and tazarotene (ethyl 6-[2-(4,4-dimethylthiochroman-6-yl)-ethynyl]nicotinate). One or more retinoids may be used herein. Preferred retinoids are retinol, retinyl palmitate, retinyl acetate, retinyl proprionate, retinal and combinations thereof. More preferred are retinol and retinyl palmitate.

The retinoid may be included as the substantially pure material, or as an extract obtained by suitable physical and/or chemical isolation from natural (e.g., plant) sources. The retinoid is preferably substantially pure, more preferably essentially pure.

The compositions of this invention may contain a safe and effective amount of the retinoid, such that the resultant composition is safe and effective for regulating skin texture. The compositions preferably contain from about 0.005% to about 2%, more preferably 0.01% to about 2%, retinoid. Retinol is preferably used in an amount of from or about 0.01% to about 0.15%; retinol esters are most preferably used in an amount of from or about 0.01% to about 2%; retinoic acids are most preferably used in an amount of from about 0.01% to about 0.25%; tocopheryl-retinoate [tocopherol ester of retinoic acid (trans- or cis), adapalene {6-[3-(1-adamantyl)-4-methoxyphenyl]-2-naphthoic acid}, and tazarotene are more preferably used in an amount of from about 0.01% to about 2%.

Another class of skin texture-regulating agent according to the present invention comprises humectants. Suitable humectants useful herein include poly-hydric alcohols, sodium 2-pyrrolidone-5-carboxylate (NaPCA), amino acids and derivatives, guanidine; glycolic acid and glycolate salts (e.g. ammonium and quaternary alkyl ammonium); lactic acid and lactate salts (e.g. ammonium and quaternary alkyl ammonium); other alpha hydroxy acids such as malic acid, aloe vera in any of its variety of forms (e.g., aloe vera gel); hyaluronic acid, precursors and derivatives thereof (e.g., glucosamine and salt derivatives such as sodium hyaluronate); lactamide monoethanolamine; acetamide monoethanolamine; urea; and mixtures thereof. Preferred for use in the compositions of the present invention are polyhydric alcohols.

Suitable polyhydric alcohols for use herein include polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, erythritol, threitol, pentaerythritol, xylitol, glucitol, mannitol, hexylene glycol, butylene glycol (e.g., 1,3-butylene glycol), hexane triol (e.g., 1,2,6-hexanetriol), trimethylol propane, neopentyl glycol, glycerine, ethoxylated glycerine and propoxylated glycerine. Preferred polyhydric alcohols of the present invention are polyhydric alcohols with 3 to 9 carbon atoms in the molecule. Examples include glycerine, butylene glycol, propylene glycol, dipropylene glycol, polyethylene glycol and derivatives thereof, hexane triol, ethoxylated glycerine and propoxylated glycerine, and mixtures thereof. More preferred for use in the present invention is glycerin. The compositions of the present invention comprise from about 5% to about 40% polyhydric alcohol, preferably from about 8% to about 15% by weight of the composition.

A further class of humectants are the amino acids and their derivatives. Suitable amino acids for use herein include both D- and L-isomers of naturally occurring amino acids. Suitable examples include L-isomers of serine, alanine, proline and hydroxyproline The topical compositions of the present invention may further comprise a thickening agent. The topical compositions of the present invention comprise from about 0.1% to about 5%, preferably from about 0.1% to about 3%, and more preferably from about 0.25% to about 2%, thickening agent by weight of the composition.

Suitable thickening agents include cellulose and derivatives such as cellulose, carboxymethyl hydroxyethylcellulose, cellulose acetate propionate carboxylate, hydroxyethyl-cellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl-substituted celluloses. In these polymers, the hydroxy groups of the cellulose polymer is hydroyxalkylated (preferably hydroxyethylated or hydroxy-propylated) to form a hydroxyalkylated cellulose which is then further modified with a C₁₀-C₃₀ straight chain or branched chain alkyl group through an ether linkage. Typically these polymers are ethers of C₁₀-C₃₀ straight or branched chain alcohols with hydroxyalkylcelluloses. Examples of alkyl groups useful herein include those selected from the group consisting of stearyl, isostearyl, lauryl, myristyl, cetyl, isocetyl, cocoyl (i.e. alkyl groups derived from the alcohols of coconut oil), palmityl, oleyl, linoleyl, linolenyl, ricinoleyl, behenyl, and mixtures thereof. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename Natrosol™ CS Plus from Aqualon Corporation.

Other useful thickeners include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof. Also useful are acrylic acid/ethyl acrylate copolymers and the carboxyvinyl polymers sold by the B.F. Goodrich Company under the trademark of Carbopol resins. Suitable Carbopol resins are described in WO98/22085.

Preferred compositions of the present invention include a thickening agent selected from carboxylic acid polymers, crosslinked polyacrylates, polyacrylamides, xanthan gum and mixtures thereof, more preferably selected polyacrylamide polymers, xanthan gum and mixtures thereof. Preferred polyacrylamides are predispersed in a water-immiscible solvent such as mineral oil and the like, containing a surfactant (HLB from about 7 to about 10) which helps to facilitate water dispersibility of the polyacrylamide. Also preferred for use herein is the non-ionic polymer under the CTFA designation: polyacrylamide and isoparaffin and laureth-7, available under the trade name Sepigel 305 from Seppic Corporation. More preferred for use herein are the co-polymer compositions commercially available from BASF Corp. under the tradename Luvigel EM™ and the co-polymer compositions available from CIBA Speciality Chemicals, Macclesfield, UK, under the tradename Salcare SC91™.

The topical compositions herein are preferably in the form of a water-in-oil or oil-in-water emulsion. Preferably the topical compositions herein are water-in-oil emulsions wherein the composition comprises one or more oil phases in an aqueous continuous phase, each oil phase comprising a single oily component or a mixture of oily components in miscible or homogeneous form. Different oil phases contain different materials, or different combinations of materials, from each other. The total level of oil phase components in the compositions of the invention is typically from about 0.1% to about 60%, preferably from about 1% to about 30%, more preferably from about 3% to about 20% and most preferably from about 5% to about 15%.

In preferred embodiments, the oil phase preferably comprises oily components such as a natural or synthetic oils selected from mineral, vegetable, and animal oils, fats and waxes, fatty acid esters, fatty alcohols, fatty acids and mixtures thereof. Preferred for use herein are for example, saturated and unsaturated fatty alcohols such as behenyl alcohol, cetyl alcohol and stearyl alcohol and hydrocarbons such as mineral oils or petrolatum.

The present compositions may further comprise a silicone phase. The silicone phase can comprise one or more silicone components such as silicone fluids, gums, and mixtures thereof. The, or each, silicone phase generally comprises from about 0.1% to about 20%, preferably from about 0.2% to about 10%, more preferably from about 0.3% to about 5%, of the composition.

Silicone components can be fluids, including straight chain, branched and cyclic silicones. Suitable silicone fluids useful herein include silicones inclusive of polyalkyl siloxane fluids, polyaryl siloxane fluids, cyclic and linear polyalkylsiloxanes, polyalkoxylated silicones, amino and quaternary ammonium modified silicones, polyalkylaryl siloxanes or a polyether siloxane copolymer and mixtures thereof. The silicone fluids can be volatile or non-volatile.

Suitable polydimethyl siloxanes that can be used herein include those available, for example, from the General Electric Company as the SF and Viscasil™ series and from Dow Corning as the Dow Corning 200 series. Also useful are essentially non-volatile polyalkyl-arylsiloxanes, for example, polymethylphenylsiloxanes, having viscosities of about 0.65 to 30,000 mm²·s⁻¹ at 25° C. These siloxanes are available, for example, from the General Electric Company as SF 1075 methyl phenyl fluid or from Dow Corning as 556 Cosmetic Grade Fluid. Cyclic polydimethylsiloxanes suitable for use herein are those having a ring structure incorporating from about 3 to about 7 (CH₃)₂SiO moieties.

Another class of silicone components suitable for use in a silicone oil phase herein includes polyether siloxane copolymers. Examples of polyether siloxan copolymers include polydiorganosiloxane-polyoxyalkylene copolymers containing at least one polydiorganosiloxane segment and at least one polyoxyalkylene segment.

The silicone components can also comprise silicone gums. The term “silicone gum” herein includes high molecular weight silicones having a weight average molecular weight in excess of about 200,000 and preferably from about 200,000 to about 4,000,000. Included are non-volatile polyalkyl and polyaryl siloxane gums. In preferred embodiments, a silicone oil phase comprises a silicone gum or a mixture of silicones including the silicone gum.

Useful herein are silicone/gum fluid blends. Preferred silicone-gum fluid blend based component for use in the compositions herein is a dimethiconol gum having a molecular weight of from about 200,000 to about 4,000,000 along with a silicone fluid carrier with a viscosity of about 0.65 to 100 mm²·s⁻¹. An example of this silicone component is Dow Corning Q2-1503 (85% 5 mm²·s⁻¹ Dimethicone Fluid/15% Dimethiconol) and Dow Coming Q2-1501 available from Dow Corning.

The topical compositions of the present invention preferably comprise emollient materials including branched chain hydrocarbons having an weight average molecular weight of from about 100 to about 15,000, preferably from about 100 to 1000; compounds of formula I:

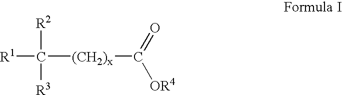

wherein R¹ is selected from H or CH₃, R², R³ and R⁴ are independently selected from C₁-C₂₀ straight chain or branched chain alkyl, and x is an integer of from 1-20; and compounds having the formula (II):

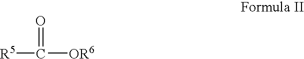

wherein R⁵ is selected from optionally hydroxy or C₁-C₄ alkyl substituted benzyl and R₆ is selected from C₁-C₂₀ branched or straight chain alkyl; and mixtures thereof.

Suitable branched chain hydrocarbons for use herein include isododecane, isohexadecane, isoeicosane, isooctahexacontane, isohexapentacontahectane, isopentacontaoctactane, and mixture thereof. Suitable for use herein are branched chain aliphatic hydrocarbons sold under the trade name Permethyl™ and commercially available from Presperse Inc., P.O. Box 735, South Plainfield, N.J. 07080, U.S.A. Suitable ester emollient materials of Formula I above include, but are not limited to, methyl isostearate, isopropyl isostearate, isostearyl neopentanoate, isononyl isononanoate, isodecyl octanoate, isodecyl isononanoate, tridecyl isononanoate, myristyl octanoate, octyl pelargonate, octyl isononanoate, myristyl myristate, myristyl neopentanoate, myristyl octanoate, myristyl propionate, isopropyl myristate and mixtures thereof. Suitable ester emollient materials of Formula (II) include but are not limited to C12-15 alkyl benzoates.

Preferred emollients for use herein are isohexadecane, isononyl isononanoate, methyl isostearate, isopropyl isostearate, and mixtures thereof. A further emollient suitable for use in the composition of the present invention is petrolatum.

The emollient material is preferably present in the compositions at a level of from about 0.1% to about 10%.

The present compositions herein may comprise an emulsifier and/or surfactant, generally to help disperse and suspend the discontinuous phase within the continuous phase. For convenience hereinafter emulsifiers will be referred to under the term ‘surfactants’, thus ‘surfactant(s)’ will be used to refer to surface active agents whether used as emulsifiers or for other surfactant purposes such as skin cleansing. Known or conventional surfactants can be used in the composition, provided that the selected agent is chemically and physically compatible with essential components of the composition, and provides the desired characteristics.

The compositions of the present invention preferably comprise from about 0.05% to about 15% of a surfactant or mixture of surfactants. The exact surfactant or surfactant mixture chosen will depend upon the pH of the composition and the other components present.

Preferred surfactants are nonionic. Among the nonionic surfactants that are useful herein are those that can be broadly defined as condensation products of long chain alcohols, e.g. C₈₋₃₀ alcohols, with sugar or starch polymers, i.e., glycosides. These compounds can be represented by the formula (S)ₙ—O—R wherein S is a sugar moiety; n is an integer of from about 1 to about 1000, and R is a C₈₋₃₀ alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples include a mixture of cetearyl glucosides and cetearyl alcohols such as those commercially available as Montanov 68™ from Seppic and Emulgade PL68/50™ available from Henkel.

Other useful nonionic surfactants include the condensation products of alkylene oxides with fatty acids (i.e. alkylene oxide esters or diesters of fatty acids). These materials have the general formula RCO(X)ₙOH or RCO(X)ₙOOCR wherein R is a C₁₀₋₃₀ alkyl group, X is —OCH₂CH₂— (i.e. derived from ethylene glycol or oxide) or —OCH₂CHCH₃— (i.e. derived from propylene glycol or oxide), and n is an integer from about 6 to about 200. Other nonionic surfactants are the condensation products of alkylene oxides with fatty alcohols (i.e. alkylene oxide ethers of fatty alcohols). These materials have the general formula R(X)ₙOR′ wherein R is a C₁₀₋₃₀ alkyl group, X is —OCH₂CH₂— or —OCH₂CHCH3—, and n is an integer from about 6 to about 100 and R′ is H or a C10-30 alkyl group. Still other nonionic surfactants are the condensation products of alkylene oxides with both fatty acids and fatty alcohols [i.e. wherein the polyalkylene oxide portion is esterified on one end with a fatty acid and etherified (i.e. connected via an ether linkage) on the other end with a fatty alcohol]. These materials have the general formula RCO(X)ₙOR′ wherein R and R′ are C₁₀₋₃₀ alkyl groups, X is —OCH₂CH₂ or —OCH₂CHCH₃—, and n is an integer from about 6 to about 100. Still other useful nonionic surfactants include polyhydroxy fatty acid amide surfactants, which are described in more detail in WO98/04241.

Other nonionic surfactants suitable for use herein include sugar esters and polyesters, alkoxylated sugar esters and polyesters, C₁-C₃₀ fatty acid esters of C₁-C₃₀ fatty alcohols, alkoxylated derivatives of C₁-C₃₀ fatty acid esters of C₁-C₃₀ fatty alcohols, alkoxylated ethers of C1-C30 fatty alcohols, polyglyceryl esters of C₁-C₃₀ fatty acids, C₁-C₃₀ esters of polyols, C₁-C₃₀ ethers of polyols, alkyl phosphates, polyoxyalkylene fatty ether phosphates, fatty acid amides, acyl lactylates, and mixtures thereof.

Another emulsifier useful herein are fatty acid ester blends based on a mixture of sorbitan or sorbitol fatty acid ester and sucrose fatty acid ester, the fatty acid in each instance being preferably C₈-C₂₄, more preferably C₁₀-C₂₀. The preferred fatty acid ester emulsifier is a blend of sorbitan or sorbitol C₁₆-C₂₀ fatty acid ester with sucrose C₁₀-C₁₆ fatty acid ester, especially sorbitan stearate and sucrose cocoate. This is commercially available from ICI under the name Arlatone 2121™.

Preferred among the nonionic surfactants are those selected from the group consisting of cetearyl glucosides, cetearyl alcohols, PEG-100 stearate, sorbitan stearate and mixtures thereof.

Emulsions of the present invention may include a silicone containing emulsifier or surfactant. A wide variety of silicone emulsifiers are useful herein. These silicone emulsifiers are typically organically modified organopolysiloxanes, also known to those skilled in the art as silicone surfactants. Useful silicone emulsifiers include dimethicone copolyols. Other examples include alkyl-modified dimethicone copolyols, i.e., compounds that contain C₂-C₃₀ pendant side chains. Still other useful dimethicone copolyols include materials having various cationic, anionic, amphoteric, and zwitterionic pendant moieties.

The composition of the present invention comprises water. Preferably, water comprises from about 30% to about 85% by weight of the composition, more preferably about 50% to about 75% by weight of the composition.

The compositions of the present invention can further comprise optional ingredients. Optional ingredients are well known in the art, and can be added without reacting with and altering the chemistry therein. Optional ingredients include additional actives, neutralizing agents, sunscreening agents and mixtures thereof.

An optional active for use in the compositions according to the present invention comprises a vitamin C compound. Vitamin C compounds include water-soluble ascorbic acid salts and esters thereof. Vitamin C compounds are particularly useful as skin lightening agents. Suitable examples include magnesium ascorbyl phosphate and the sodium salt of the monophosphate ester of ascorbic acid, commercially available from Roche Vitamins Europe Ltd as Stay-C50™.

Another optional ingredient is neutralizing agents. Neutralizing agents suitable for use in neutralizing acidic groups containing hydrophilic gelling agents herein include sodium hydroxide, potassium hydroxide, ammonium hydroxide, monoethanolamine, diethanolamine, amino methyl propanol, tris-buffer and triethanolamine.

A further optional component may comprise sunscreening agents. Preferred among those sunscreens which are useful in the compositions of the invention are those selected from 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-butyldibenzoylmethane, 4-isopropyl dibenzoylmethane, 3-benzylidene camphor, 3-(4-methylbenzylidene) camphor, titanium dioxide, zinc oxide, silica, iron oxide, Parsol MCX, Eusolex 6300, Octocrylene, Parsol 1789, and mixtures thereof.

Generally, the sunscreens can comprise from about 0.5% to about 20% of the compositions useful herein. Exact amounts will vary depending upon the sunscreen chosen and the desired Sun Protection Factor (SPF). SPF is a commonly used measure of photoprotection of a sunscreen against erythema. See Federal Register, Vol. 43, No. 166, pp. 38206-38269, Aug. 25, 1978.

Further optional ingredients include oil-soluble actives. Suitable oil soluble actives for use herein include vitamin E and its derivatives, salicylic acid and other beta-hydroxy acids, perfumes and occlusion materials, and mixtures thereof.

### EXAMPLES

The following examples further describe and demonstrate embodiments within the scope of the present invention. They are given for the purpose of illustration and are not to be construed as limitations of the present invention. Where applicable, ingredients are given in CTFA name.

### Examples I to V

| | I | II | III | IV | V |
|---|---|---|---|---|---|
| INGREDIENTS | % w/w | % w/w | % w/w | % w/w | % w/w |
| DEIONISED WATER | QS | QS | QS | QS | QS |
| DISODIUM EDTA | 0.1 | 0.1 | 0.1 | 0.1 | 0.1 |
| GLYCERIN | 10.0 | 15.0 | 10.0 | 25.0 | 10.0 |
| NIACINAMIDE | 3.5 | 3.50 | 5.0 | 5.0 | 3.5 |
| PANTHENOL | 0.5 | 1.0 | 1.0 | 1.0 | 0.5 |
| EMULGADE¹ | 0.2 | 0.2 | 0.2 | 0.2 | 0.2 |
| ISOHEXADECANE | 3.0 | 6.0 | 6.0 | 2.0 | — |
| ETHYL PARABEN | 0.15 | 0.15 | 0.15 | 0.15 | 0.15 |
| COCONUT OIL | — | — | 0.2 | 3.0 | — |
| FRACTIONATED | | | | | |
| PROPYL PARABEN | 0.07 | 0.07 | 0.07 | 0.07 | 0.07 |
| STEARIC ACID | 0.1 | 0.1 | 0.1 | 0.1 | 0.1 |
| PEG-100 STEARATE² | 0.1 | 0.1 | 0.1 | 0.1 | 0.1 |
| STEARYL ALCOHOL | 0.61 | 0.61 | 0.61 | 0.61 | 0.61 |
| CETYL ALCOHOL | 0.49 | 0.49 | 0.49 | 0.49 | 0.49 |
| BEHENYL ALCOHOL | 0.40 | 0.40 | 0.40 | 0.40 | 0.40 |
| ISOPROPYL | 1.5 | 3.0 | 1.5 | 1.5 | 0.65 |
| ISOSTEARATE³ | | | | | |
| DL-α TOCOPHEROL | 0.25 | 0.5 | 0.5 | 0.5 | 0.25 |
| ACETATE | | | | | |
| MINERAL OIL | — | 1.0 | — | — | — |
| PETROLATUM | 2.0 | — | 1.5 | 1.5 | 0.5 |
| LUVIGEL EM⁴ | — | — | 2.0 | 2.0 | 2.0 |
| SEPIGEL 305⁵ | 1.50 | 1.50 | — | — | — |
| RHODASURF L7⁶ | 0.0055 | 0.0055 | 0.0055 | 0.0055 | 0.0055 |
| VOLPO 3⁷ | 0.045 | 0.045 | 0.045 | 0.045 | 0.045 |
| SODIUM | 0.011 | 0.011 | 0.011 | 0.011 | — |
| HYDROXIDE | | | | | |
| NYLONPOLY WL 10⁸ | 1.0 | 1.0 | 1.0 | 1.0 | 1.0 |
| DRY FLO PLUS⁹ | 1.0 | 1.5 | 2.0 | 2.0 | 1.0 |
| BENZYL ALCOHOL | 0.25 | 0.25 | 0.25 | 0.25 | 0.25 |
| DC 1503¹⁰ | 1.0 | 1.5 | 1.5 | 1.5 | 2.0 |
| PERFUME | 0.3 | 0.3 | 0.3 | 0.3 | 0.3 |
| OCTOCRYLENE | — | — | — | — | 1.0 |
| SILVER PEARL¹² | 0.8 | 0.6 | 0.3 | 0.6 | 0.8 |
| GOLD PEARL¹² | 0.2 | 0.4 | 0.2 | 0.4 | 0.2 |
| AVOBENZONE | — | — | — | — | 1.50 |
| OCTYL SALICYLATE | — | — | — | — | 3.5 |
| PHENYL | — | — | — | — | 1.0 |
| BENZIMIDAZOLE | | | | | |
| SULPHONIC ACID | | | | | |
| TRIETHANOL-AMINE | — | — | — | — | 0.61 |

¹Emulgade: Supplier: Cognis Deutchland GmbH, Paul-Thomas Strasse 56, D-40551 Dusseldorf, Germany.
²PEG 100 Stearate supplied by ICI, PO Box 90, Wilton Centre, Middlesborough, Cleveland, England. TS6 8JE
³Volpo L3; Supplied by Scher Chemicals Inc, Industrial West, Clifton, NJ 07012
⁴Luvigel EM: Supplier: BASF Plc, PO Box 4-Earl Road, Cheadle Hulme, Cheshire SK8 6QG
⁵Sepigel 305: Supplier: Seppic, 75 Quai D'Orsay, Paris
⁶Rhodasurf: Supplier: Coldic, Staisty Close, Homewood Trading Estate, Chesterfield, Derbyshire S42 5UG
⁷Volpo 3: Supplier: Croda Oleochemicals, Cowick hall, Snaith Goole, East Yorkshire, Dn14 9AA
⁸Nylonpoly: Supplier: Optima Chemicals, Unit 17, Chiltern Business Village, Arundel Road, Uxbridge, Middlesex, UB8 2SN
⁹Dry Flo: Supplier: National Starch Chemical Company, 10, Finderne Avenue, Bridgewater, NJ 08807, USA
^{10&11}DC 1503, DC9040: Supplied by Dow Coming, Kings Court, 185 Kinds Rd, Reading, Berks, RGI 4EX
¹²Silver and Gold ‘Prestige’ pearls: Supplier: Eckart Gmbh and Co., Kaiserstrasse 30, 90763 Fuerth, Germany.

### Examples VI to IX

| | VI | VII | VIII | IX |
|---|---|---|---|---|
| INGREDIENTS | % w/w | % w/w | % w/w | % w/w |
| DEIONISED | QS | QS | QS | QS |
| WATER | | | | |
| DISODIUM EDTA | 0.1 | 0.1 | 0.1 | 0.1 |
| GLYCERIN | 10.0 | 10.0 | 10.0 | 15.0 |
| NIACINAMIDE | 3.5 | 3.5 | 3.5 | 3.5 |
| PANTHENOL | 0.5 | 0.5 | 0.5 | 0.5 |
| EMULGADE¹ | 0.2 | 0.2 | 0.2 | 0.2 |
| ISOHEXADECANE | 3.0 | 6.0 | 6.0 | 3.0 |
| ETHYL PARABEN | 0.15 | 0.15 | 0.15 | 0.15 |
| COCONUT OIL | — | — | — | — |
| FRACTIONATED | | | | |
| PROPYL | 0.07 | 0.07 | 0.07 | 0.07 |
| PARABEN | | | | |
| STEARIC ACID | 0.1 | 0.1 | 0.1 | 1.0 |
| PEG-100 | 0.1 | 0.1 | 0.1 | 0.1 |
| STEARATE² | | | | |
| STEARYL | 0.61 | 0.61 | 0.61 | 0.79 |
| ALCOHOL | | | | |
| CETYL ALCOHOL | 0.49 | 0.49 | 0.49 | 0.64 |
| BEHENYL | 0.40 | 0.40 | 0.40 | 0.52 |
| ALCOHOL | | | | |
| ISOPROPYL | 1.5 | 3.0 | 1.5 | 1.5 |
| ISOSTEARATE³ | | | | |
| DL-α | 0.25 | 0.5 | 0.5 | 0.25 |
| TOCOPHEROL | | | | |
| ACETATE | | | | |
| MINERAL OIL | — | 1.0 | — | — |
| PETROLATUM | 2.0 | — | 1.5 | 2.0 |
| LUVIGEL EM⁴ | 2.0 | — | — | 0.5 |
| SEPIGEL 305⁵ | — | 1.5 | 1.5 | — |
| RHODASURF L7⁶ | 0.0055 | 0.0055 | 0.0055 | 0.0055 |
| VOLPO 3⁷ | 0.045 | 0.045 | 0.045 | 0.045 |
| SODIUM | 0.011 | 0.011 | 0.011 | 0.011 |
| HYDROXIDE | | | | |
| NYLONPOLY WL | 1.0 | 1.0 | 1.0 | 1.0 |
| 10⁸ | | | | |
| DRY FLO PLUS⁹ | 1.5 | 1.5 | 1.5 | 1.0 |
| BENZYL | 0.25 | 0.25 | 0.25 | 0.25 |
| ALCOHOL | | | | |
| DC 1503¹⁰ | — | 1.5 | 1.5 | 1.0 |
| PERFUME | 0.3 | 0.3 | 0.3 | 0.3 |
| DC 9040¹¹ | 5.0 | — | — | — |
| SILVER PEARL¹² | 0.8 | 0.6 | 0.6 | 0.3 |
| GOLD PEARL¹² | 0.2 | 0.4 | 0.4 | 0.2 |
| TITANIUM | — | 1.0 | 1.0 | — |
| DIOXIDE | | | | |
| IRON OXIDES | — | 0.11 | — | — |
| FD&C YELLOW 5 | — | — | 0.005 | — |
| FD&C RED 40 | — | — | 0.002 | — |

The compositions are made as follows:

A water phase is prepared by admixing all water soluble ingredients, except sodium hydroxide and panthenol, in water and heating to about 800C. A second premix is prepared by admixing of the oil soluble ingredients except the silicone oil (DC1503) and heating also to around 800C. The oil phase is added to the water phase and sheared to form an emulsion.

The emulsion is cooled to 600C and the polymeric thickener (Luvigel EM) and associated anionic surfactants (oleth 3, laureth 7)are then added. At 55degC., sodium hydroxide solution is then added to neutralise to pH 6-7.5, except for examples where sunscreens are included. At 45-500C. the panthenol, benzyl alcohol, DC1503, dyes and particles (including mica pearls, titanium dioxide and iron oxides) are added and the resulting product is sheared to ensure particle dispersion, de-agglomeration and homogeneity. The composition can then be cooled to 40° C. and perfume can be added. The product can then be prepared for packaging.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A topical composition comprising gold-coloured particulates and silver-coloured particulates wherein the composition, when measured at 15° from specular 15 minutes after topical application of 2□l/cm² in vivo, has a reflectance of less than about 200 and a delta E value of less than about 4.

2. The cosmetic composition according to claim 1 having a reflectance of from about 25 to about 60.

3. The cosmetic composition according to claim 1 having a delta E value of from about 1.5 to about 2.5.

4. The cosmetic composition according to claim 1 wherein the ratio of gold-coloured particulates to silver-coloured particulates is from about 5:1 to about 1:5.

5. The cosmetic composition according to claim 1 comprising from about 0.1% to about 5% gold-coloured particulates and from about 0.1% to about 5% silver-coloured particulates.

6. The cosmetic composition according to claim 1 wherein the gold-coloured particulates comprise TiO₂.

7. The cosmetic composition according to claim 1 wherein the silver-coloured particulates comprise TiO₂.

8. The cosmetic composition according to claim 1 wherein the gold-coloured particulates comprise SnO.

9. The cosmetic composition according to claim 1 wherein the silver-coloured particulates comprise SnO.

10. The cosmetic composition according to claim 1 wherein the gold-coloured particulates have a diameter of from about 5 microns to about 75 microns.

11. The cosmetic composition according to claim 1 wherein the silver-coloured particulates have a diameter of from about 5 microns to about 75 microns.

12. The cosmetic composition according to claim 1 wherein the gold-coloured particulates comprise a substrate with at least one interference layer there upon, said interference layer comprising TiO₂, SnO, and mixtures thereof.

13. The cosmetic composition according to claim 1 wherein the silver-coloured particulates comprise a substrate with at least one interference layer there upon, said interference layer comprising TiO₂, SnO, and mixtures thereof.

14. The cosmetic composition according to claim 12 wherein the substrate comprises platelets.

15. The cosmetic composition according to claim 12, wherein the substrate comprises mica.

16. The cosmetic composition according to claim 1 wherein the composition further comprises a skin texture-regulating agent.

17. The cosmetic composition according to claim 16, wherein the skin texture-regulating agent is selected from the group consisting of vitamin B₃ derivatives, panthenol and its derivatives, retinoids and their derivatives, humectants, amino acids and their derivatives, and mixtures thereof.

18. The cosmetic composition according claim 17 wherein the composition comprises a vitamin B₃ derivative.

19. The cosmetic composition according to claim 1 where in the composition comprises from 0.1% to 5% of a thickening agent.

20. The cosmetic composition according to claim 1 wherein the composition is in the form of an oil-in-water or water-in-oil emulsion.

21. The cosmetic composition according to claim 20 wherein the composition is in the form of an oil-in-water emulsion.

22. The cosmetic composition according to claim 13 wherein the substrate comprises platelets.

23. The cosmetic composition according to claim 13, wherein the substrate comprises mica.
